(19) United States
(12) Patent Application Publication
(10) Pub. No.: US 2006/0241674 A1
(43) Pub. Date: Oct. 26, 2006

(54) Medical insert device and associated method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/389,856

(22) Filed: Mar. 27, 2006

Related U.S. Application Data
(60) Provisional application No. 60/674,075, filed on Apr. 22, 2005.

(51) Int. Cl. A61M 29/00

(52) U.S. Cl. 606/198

(57) ABSTRACT

A medical method utilizes a tubular member. The method comprises inserting the tubular member through a patient's mouth into the patient's esophagus so that at least a portion of the tubular member is disposed in the patient's esophagus as a liner, and subsequently inserting flexible endoscopic surgical or diagnostic instruments through the patient's mouth and the tubular member in the esophagus into the patient's stomach. The tubular member is removed from the patient's esophagus after termination of the procedure.

### CROSS-REFERENCE TO RELATED APPLICATION

This application claims the benefit of U.S. Provisional Patent Application No. 60/674,075 filed Apr. 22, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures, such as trauma to the hollow internal organs through which the endoscopic instruments are passed to the target surgical site, generally but not exclusively within the abdominal cavity.

The procedures of U.S. Pat. Nos. 5,297,536 and 5,458,131 may be termed trans-organ procedures insofar as surgical operations are conducted via organs that are otherwise not implicated in the procedures.

### OBJECTS OF THE INVENTION

It is an object of the present invention to provide improvements on the afore-described surgical procedures.

It is another object of the present invention to provide a method and/or an associated device for protecting a passageway in an internal hollow organ during a trans-organ procedure.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A medical method comprises, in accordance with the present invention, providing a tubular member, inserting the tubular member through a patient's mouth into the patient's esophagus so that at least a portion of the tubular member is disposed in the patient's esophagus as a liner, and subsequently inserting flexible endoscopic surgical or diagnostic instruments through the patient's mouth and the tubular member in the esophagus into the patient's stomach. The tubular member is removed from the patient's esophagus after termination of the procedure.

Pursuant to another feature of the present invention, the inserting of the tubular member into the patient's esophagus includes providing a flexible deployment tube containing the tubular member in a collapsed configuration, inserting at least a distal end portion of the deployment tube through the patient's mouth into the patient's esophagus, ejecting the tubular member from a distal end of the deployment tube, and subsequently expanding the tubular member from the collapsed configuration to an expanded configuration inside the patient's esophagus.

The tubular member may include a frame made of a shape-memory material; the expanding of the tubular member then occurs automatically upon ejecting of the tubular member from the deployment tube.

Typically, the method further comprises removing the deployment tube from the patient's esophagus after the ejecting of the tubular member and prior to the inserting of the endoscopic instruments.

In a trans-organ surgical procedure, distal end portions of the surgical instruments are moved through at least one incision or perforation formed in a digestive tract of the patient.

A surgical kit in accordance with the present invention comprises at least one surgical instrument having an elongate flexible shaft having a length longer than a human adult esophagus, and a tubular member insertable through a patient's mouth so as to be disposed at least partially as a liner in the patient's esophagus. The tubular member has an expanded configuration and an at least partially collapsed insertion configuration, the expanded configuration having an inner diameter larger than an outer diameter of the flexible shaft so as to enable passage of a distal end portion of the shaft through the tubular member in the expanded configuration.

Pursuant to another feature of the present invention, the surgical kit further comprises a flexible deployment tube containing the tubular member in the collapsed configuration. At least a distal end portion of the deployment tube is insertable through the patient's mouth into the patient's esophagus. An ejector is disposable at least partially inside the deployment tube for ejecting the tubular member from a distal end of the deployment tube. The tubular member is expandable from the collapsed configuration to the expanded configuration inside the patient's esophagus.

The tubular member may include a frame made of a shape-memory material, so that the tubular member expands automatically upon ejection from the deployment tube.

A surgical tool may be provided having an elongate flexible shaft with an operative tip for forming at least one incision or perforation in a digestive tract of the patient. This tool is used after deployment of the esophageal liner.

An esophageal liner in accordance with the present invention comprises a tubular member having an expandable frame covered with a protective web material. The web material may include wire mesh and/or a film material.

### BRIEF DESCRIPTION OF THE DRAWINGS

FIG. 1 is a schematic perspective view of an esophageal liner in accordance with the present invention.

FIG. 2 is a partial cross-sectional view of the esophageal liner of FIG. 1.

FIG. 3 is a schematic cross-sectional view of a person's upper digestive tract.

FIG. 4 is a schematic cross-sectional view similar to FIG. 3, showing endoscopic instruments inserted in a trans-organ procedure pursuant to the teachings of U.S. Pat. Nos. 5,297,536 and 5,458,131.

FIGS. 5A-5D are schematic cross-sectional views of a person's upper digestive tract, showing successive steps in an endoscopic procedure utilizing the esophageal liner of FIG. 1, in accordance with the present invention.

FIGS. 6A and 6B are schematic perspective views of an endoscope provided with an inflatable sheath for protecting the esophagus during an endoscopic procedure, respectively showing the sheath in a deflated and an expanded configuration.

FIG. 7 is a schematic perspective view of the endoscope and the expanded sheath of FIG. 6B, showing the endoscope and sheath disposed in or traversing a person's esophagus.

FIGS. 8A-8C are schematic perspective views showing successive steps in the utilization of an esophageal liner in the form of an inflatable balloon, pursuant to the present invention.

### DETAILED DESCRIPTION

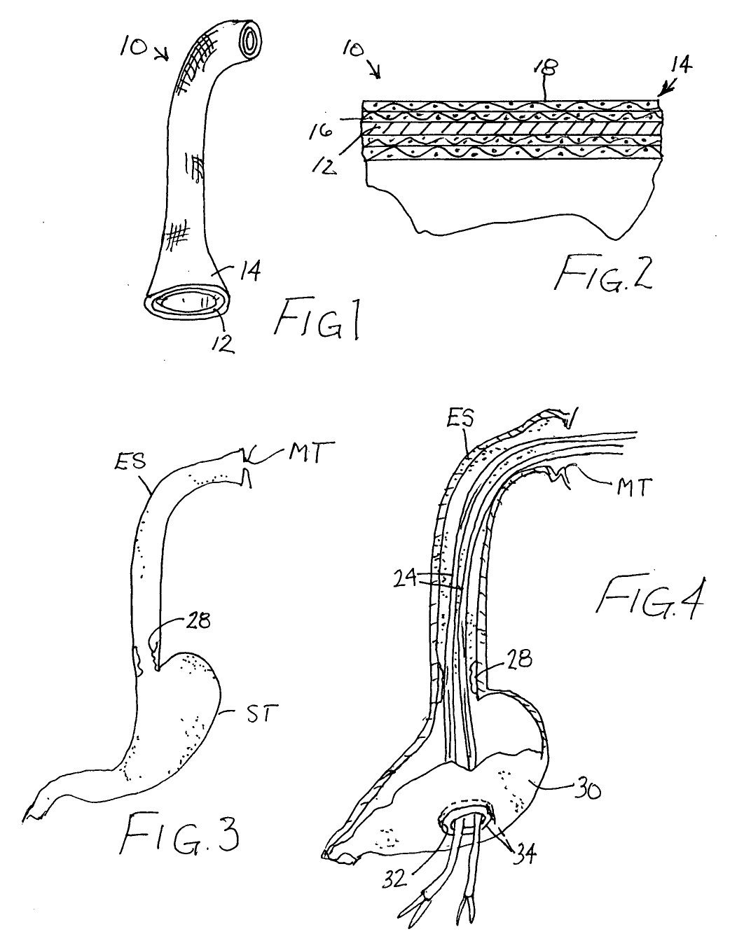

As illustrated in FIGS. 1 and 2, an esophageal liner 10 comprises a tubular member having an expandable frame 12 covered with a protective web material 14. The web material 14 may include wire mesh 16 and/or a film or fabric material 18. The web material may be disposed along an inner side as well as an outer side of frame 12.

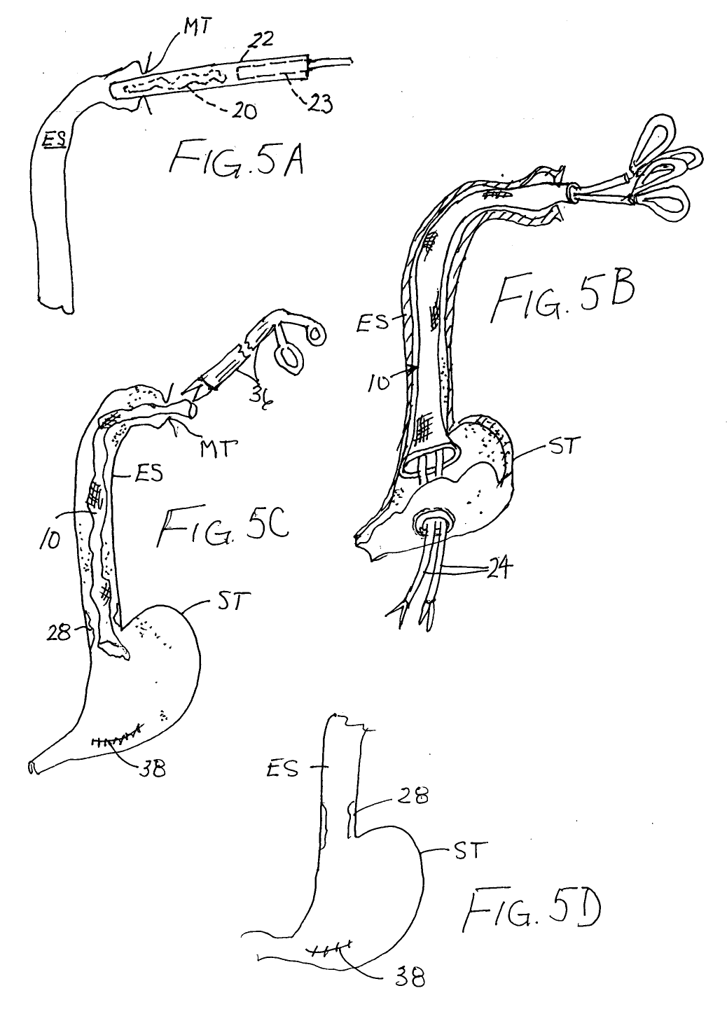

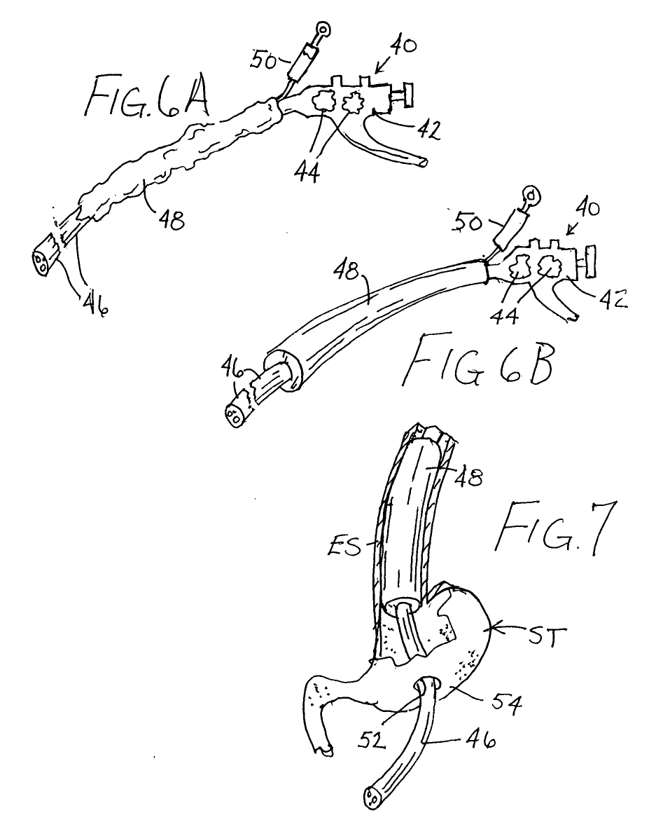

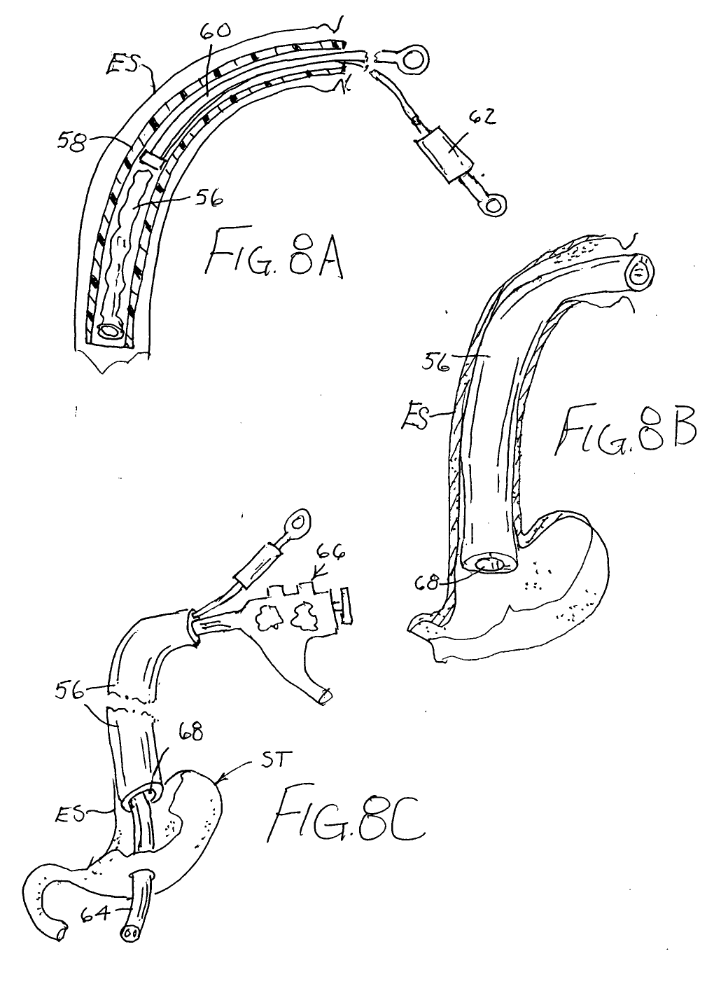

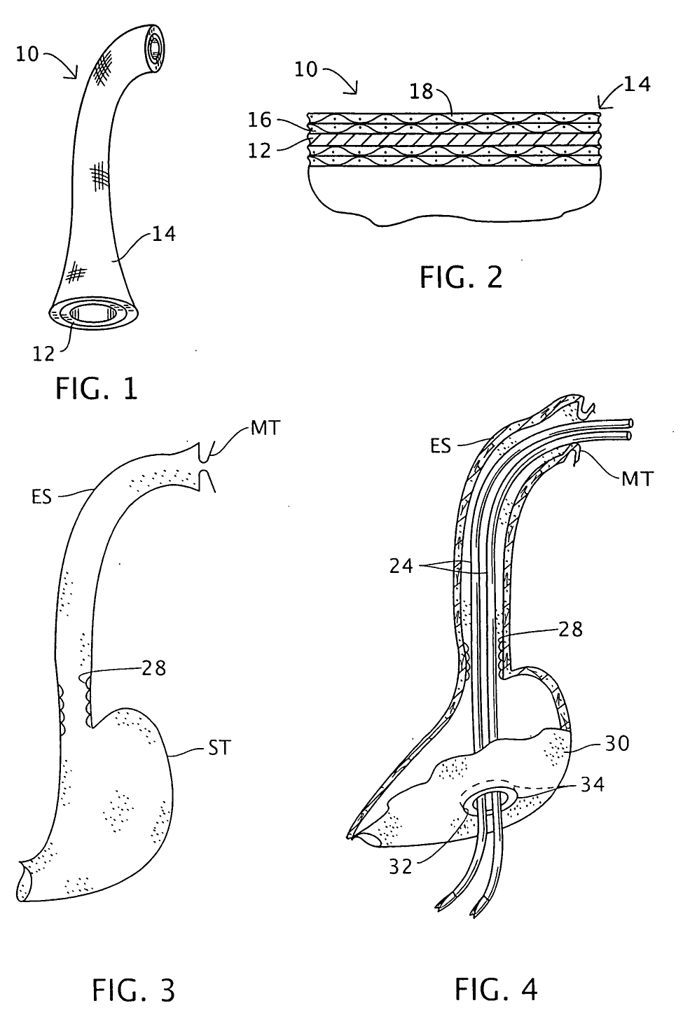

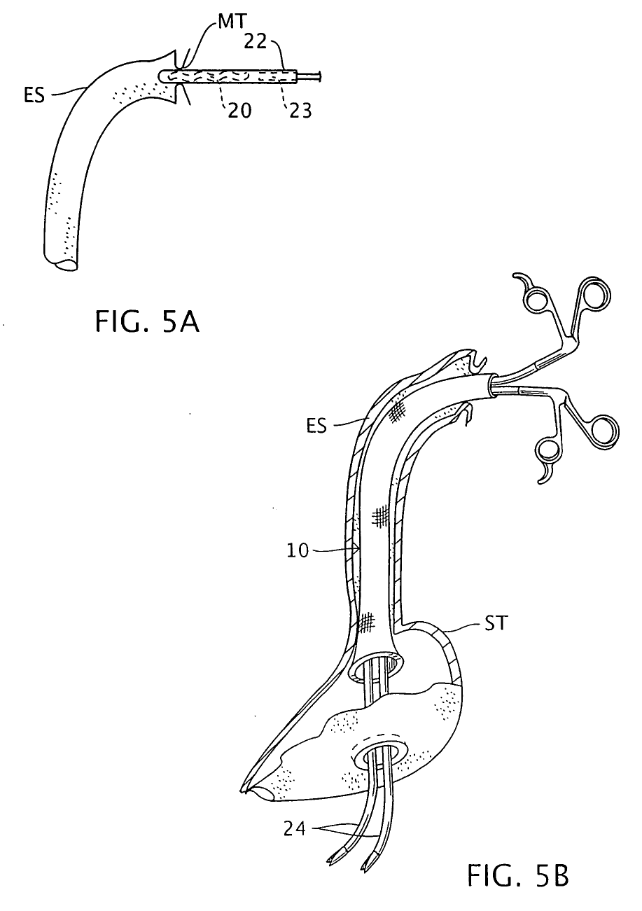

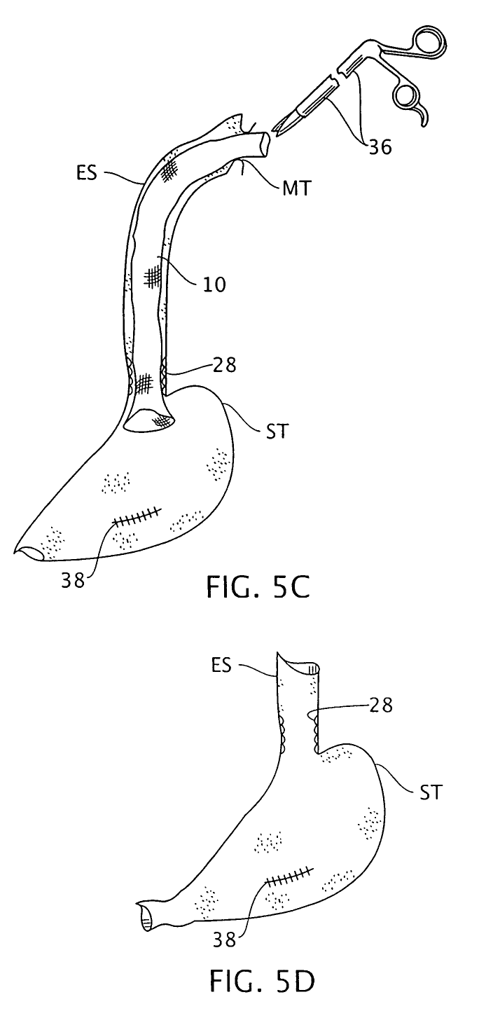

Frame 12 is made at least in part of a shape-memory material such as Nitinol that is deformable to a collapsed configuration so that liner 10 may be disposed in a collapsed configuration 20 inside a flexible deployment tube 22 (FIG. 5A).

In a trans-organ procedure as described in U.S. Pat. Nos. 5,297,536 and 5,458,131, flexible endoscopic instruments 24 are inserted through a patient's mouth MT, past the soft tissues 26 and possible varices 28 of a patient's esophagus ES and into the patient's stomach ST (FIG. 3). As illustrated in FIG. 4, the stomach wall 30 is incised to form a perforation 32, which is provided with a port element 34. Distal end portions of the instruments 24 are then passed through the port element 34 and consequently the perforation 32 into the abdominal cavity AC.

Instruments 24 can damage the soft tissues 26 and varices 28 of the esophagus ES during this procedure. However, this damage can be obviated or reduced through the use of the liner 10 of FIG. 1. At the onset of a trans-organ procedure through a patient's upper GI tract, as described in U.S. Pat. Nos. 5,297,536 and 5,458,131, deployment tube 22 is inserted into a patient's esophagus ES (FIG. 5A). Liner 10 is then ejected from tube 22 into the esophagus ES by a forward or distally directed movement of a plunger or pusher member 23 and permitted to expand from the collapsed configuration 20 to the expanded use configuration. Owing to the shape-memory material (e.g., Nitinol), the tubular frame 12 automatically expands upon ejecting of the collapsed liner 20 from deployment tube 22. In the expanded configuration (FIG. 5B), liner 10 protects the esophagus ES from being damaged by endoscopic instruments 24 inserted through the esophagus and stomach ST during a trans-organ procedure wherein distal end portions of the instruments 24 are passed through an incision or perforation 32 formed in the stomach wall 30.

Deployment tube 22 is removed from esophagus after the ejection of liner 10 and prior to the insertion of instruments 24. At the end of the procedure, perforation 32 is closed as indicated at 38 in FIGS. 5C and 5D. The liner 10 is removed from the patient's esophagus ES. As illustrated in FIG. 5C, a grasper 36 may be used to pull the liner 10 from the esophagus ES through the mouth MT.

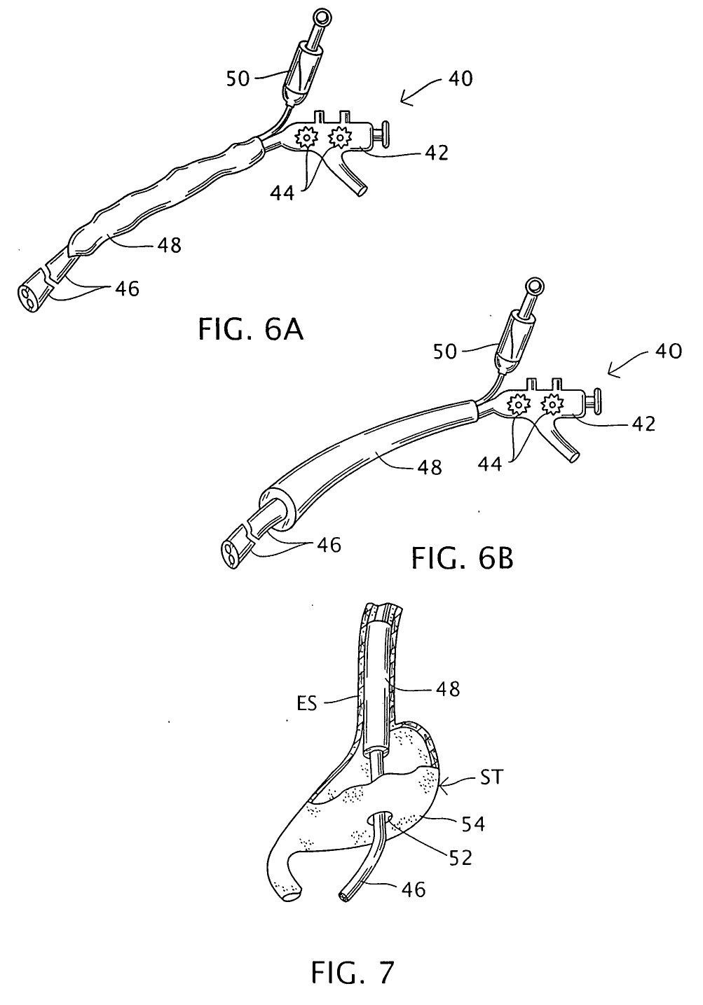

As depicted in FIGS. 6A, 6B and 7, an upper GI endoscope 40 with a hand piece 42 having directional control knobs 44 has a flexible insertion member 46 to which a sheath 48 is removably attachable. Sheath 48 particularly takes the form of an elongate annular balloon with a deflated configuration shown in FIG. 6A and an expanded configuration shown in FIGS. 6B and 7. After attachment of sheath 48 to insertion member 46, the insertion member and the sheath, in a deflated configuration, are inserted into the esophagus ES of a patient. Upon a sufficient degree of insertion, a pressure source such as a liquid-filled syringe 50 is operated to pressurize and inflate the balloon 48 to an expanded configuration (FIGS. 6B and 7). An incising instrument (not shown) may be inserted through a biopsy or working channel of endoscope 40 and manipulated from outside the patient to form an opening 52 in a wall 54 of the patient's stomach ST. Thereupon, the distal end portion (not separately enumerated) of endoscope insertion member 46 is passed through opening 52 to view organs in the patient's abdominal cavity (not illustrated). It may be necessary in some cases to deflate balloon sheath 48 to permit a repositioning of endoscope insertion member 46. After completion of a trans-gastric procedure, opening 52 is closed (see FIGS. 5C, 5D) and balloon sheath 48 is deflated and withdrawn from the esophagus ES, together with endoscope insertion member 46.

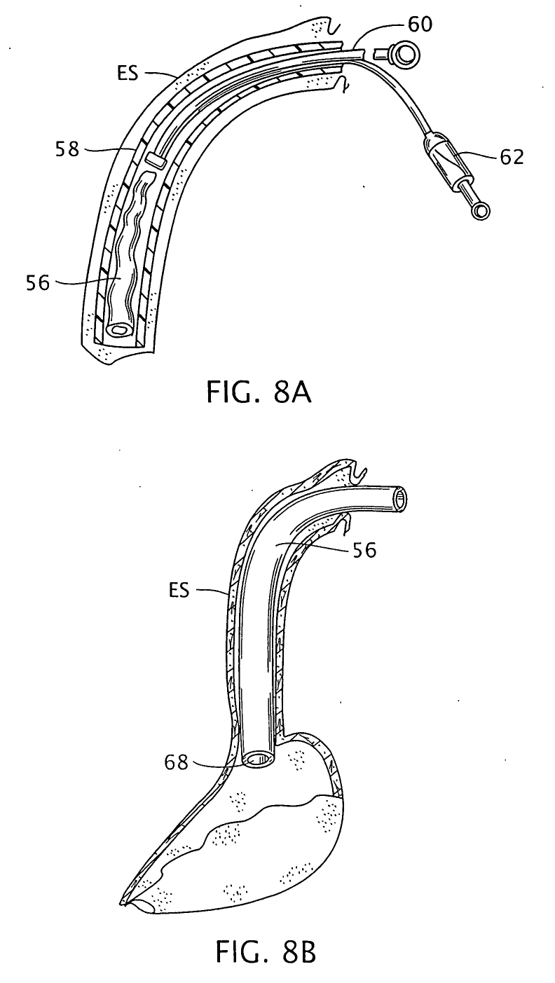

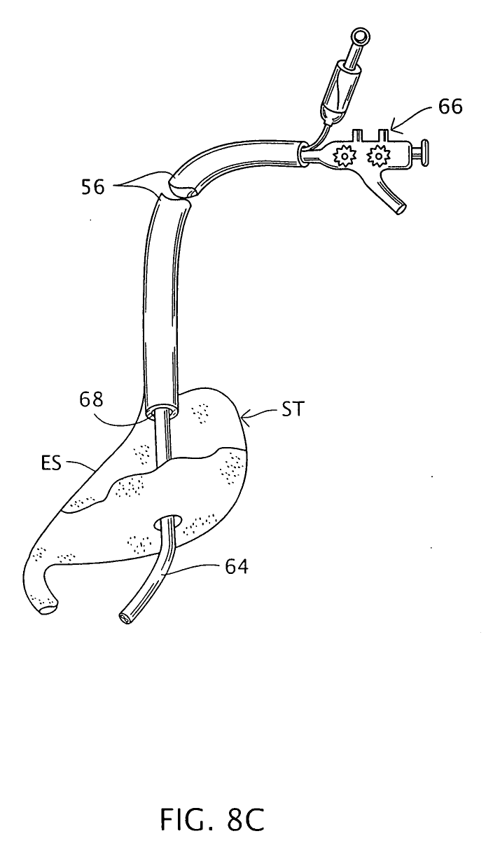

As depicted in FIG. 8A, an esophageal liner may take the form of a balloon 56 initially disposed in a collapsed configuration inside a distal end portion of a flexible deployment tube 58. Upon insertion of the distal end portion of the deployment tube 58 into an esophagus ES, a plunger or push rod 60 is moved in the distal direction to eject the deflated balloon 56 from the deployment tube and into the esophagus ES. Then deployment tube 58 is withdrawn from the patient and a pressure source such as a liquid-filled syringe 62 is actuated to inflate the balloon 56 into an expanded annular configuration shown in FIG. 8B. An insertion member 64 of an endoscope 66 is then passed through a lumen 68 of the inflated balloon liner member 58, as shown in FIG. 8C. Lumen 68 may be coated with a lubricant to facilitate sliding of the endoscope insertion member 64 in alternate directions along the esophagus ES

Various instruments and devices disclosed herein may be packaged as surgical kits that facilitate the delivery, organization and use of the instruments and devices. Such kits may comprise at least one surgical instrument 24 (FIG. 4) having an elongate flexible shaft with a length longer than a human adult esophagus ES, as sell as esophageal liner 10 or 56 or sheath 48. Liner 10 or 58 or sheath 48 has an expanded configuration and an at least partially collapsed insertion configuration, the expanded configuration having an inner diameter sufficiently large as to enable passage of a distal end portion of the shaft through the liner or sheath in the expanded configuration thereof. The surgical kits may further comprise flexible deployment tube 22 or 58, including ejector rod 23 or 60, respectively. A surgical tool such as a scalpel may be provided having an elongate flexible shaft with an operative tip in the form of a cutting blade or incising element for forming at least one incision or perforation in a digestive tract of the patient. This tool is used after deployment of the esophageal liner.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are profferred by way of example to facilitate comprehension of the invention and should not be construed to limit the scope thereof.

What is claimed is:

1. A medical method comprising:
providing a tubular member;
inserting said tubular member through a patient's mouth into the patient's esophagus so that said tubular member is disposed at least partially as a liner in the patient's esophagus; and
subsequently inserting flexible endoscopic instruments through the patient's mouth and said tubular member in the patient's esophagus into the patient's stomach.

2. The method defined in claim 1 wherein the inserting of said tubular member includes providing a flexible deployment tube containing said tubular member in a collapsed configuration; inserting at least a distal end portion of said deployment tube through the patient's mouth into the patient's esophagus; ejecting said tubular member from a distal end of said deployment tube; and subsequently expanding said tubular member from said collapsed configuration to an expanded configuration inside the patient's esophagus.

3. The method defined in claim 2 wherein said tubular member includes a frame made of a shape-memory material, the expanding of said tubular member occurring automatically upon ejecting of said tubular member from said deployment tube.

4. The method defined in claim 2, further comprising removing said deployment tube from the patient's esophagus after the ejecting of said tubular member and prior to the inserting of said endoscopic instruments.

5. The method defined in claim 1, further comprising moving distal end portions of said endoscopic instruments through at least one incision or perforation formed in a digestive tract of the patient.

6. The method defined in claim 1, further comprising removing said tubular member from the patient's esophagus after termination of a medical procedure.

7. The method defined in claim 1 wherein the tubular member extends out through the patient's mouth upon disposition of said tubular member in the patient's esophagus.

8. An esophageal liner comprising a tubular member having an expandable frame covered with a protective web material.

9. The liner defined in claim 8 wherein said web material includes wire mesh.

10. The liner defined in claim 8 wherein said web material includes a film material.

11. A surgical kit comprising:
at least one surgical instrument having an elongate flexible shaft having a length longer than a human adult esophagus; and
a tubular member insertable through a patient's mouth so as to be disposed at least partially as a liner in the patient's esophagus, said tubular member having an expanded configuration and an at least partially collapsed insertion configuration, said expanded configuration having an inner diameter sufficiently large as to enable passage of a distal end portion of said shaft through said tubular member in said expanded configuration.

12. The surgical kit defined in claim 11 wherein said tubular member includes a balloon member.

13. The surgical kit defined in claim 12 wherein said balloon member defines an elongate annular inflation chamber.

14. The surgical kit defined in claim 12 wherein said balloon member is provided along an inner surface with a lubricant.

15. The surgical kit defined in claim 11, further comprising a flexible deployment tube containing said tubular member in said collapsed configuration, at least a distal end portion of said deployment tube being insertable through the patient's mouth into the patient's esophagus, also comprising an ejector disposable at least partially inside said deployment tube for ejecting said tubular member from a distal end of said deployment tube, said tubular member being expandable from said collapsed configuration to said expanded configuration inside the patient's esophagus.

16. The surgical kit defined in claim 15 wherein said tubular member includes a frame made of a shape-memory material, so that said tubular member expands automatically upon ejection from said deployment tube.

17. The surgical kit defined in claim 11, further comprising a surgical tool with an elongate flexible shaft with an operative tip for forming at least one incision or perforation in a digestive tract of the patient.
